(19) United States
(12) Patent Application Publication
(10) Pub. No.: US 2022/0328159 A1
(43) Pub. Date: Oct. 13, 2022

(54) RANGE OF MOTION DETERMINATION

(71) Applicant: PeerWell, Inc., San Francisco, CA (US)

(72) Inventors: Jeffrey Miles Greenberg, San Francisco, CA (US); Renhao Wang, Toronto (CA); Manish Shah, San Francisco, CA (US); Borja Arias Drake, Rijeka (HR); Emmett Jackson Greenberg, Mill Valley, CA (US); Oriol Janés Pereira, Barcelona (ES)

(21) Appl. No.: 17/717,074

(22) Filed: Apr. 9, 2022

Related U.S. Application Data
(60) Provisional application No. 63/173,340, filed on Apr. 9, 2021.

(51) Int. Cl. G16H 20/30

(52) U.S. Cl. CPC G16H 20/30

(57) ABSTRACT

Various embodiments of an apparatus, methods, systems and computer program products described herein are directed to a Movement Conformance Engine. The Movement Conformance Engine registers a physical location of a computing device with respect to a user in a three-dimensional (3D) space. The Movement Conformance Engine collects movement data of the computing device from the registered location during performance of a predefined movement by the user. The Movement Conformance Engine determines an action based on at least an attribute associated with the movement data.

### CROSS-REFERENCE TO RELATED APPLICATIONS

This application is a Non-Provisional application of, and claims the benefit of U.S. Patent Application No. 63/173,340, filed Apr. 9, 2021, which is hereby incorporated by reference in its entirety.

### SUMMARY

Conventional systems are deficient with respect to tracking a user's performance of one or more predefined movements and generating feedback for the user during performance of a predefined movement(s) and further determining a rate and/or degree of improvement in the user's subsequent performances of the predefined movement(s).

Various embodiments of a Movement Conformance Engine provide for significant improvements and advantages over conventional systems with regard to such tracking and generating feedback.

Various embodiments of an apparatus, methods, systems and computer program products described herein are directed to a Movement Conformance Engine. According to various embodiments, the Movement Conformance Engine captures data associated with a user's physical movements and generates machine learning input based on the captured data. The Movement Conformance Engine feeds the machine learning input into one or more machine learning models. The Movement Conformance Engine receives machine learning output that represents whether the user's performance of the physical movements conforms to an expected performance of the physical movements. In addition, the machine learning output may represent a comparison of the user's performance of the physical movements with various types of segments of other users. The machine learning output may further represent a rate of improvement of the user, and thereby reflect an improvement (or deterioration) in the user's health/physical condition.

Various embodiments of the Movement Conformance Engine determine range of motion measures for one or more movements performed by the user in three dimensions rather than the conventional practice which is limited to two dimensions. Given the 3D position, the Movement Conformance Engine projects back into various skeletal and muscular components involved in respective movement and pain and/or performance measurement can be determined at any one point in the musculoskeletal complex.

Various embodiments of the Movement Conformance Engine may implement a measurement pipeline that selects and/or determines a physical orientation of a computing device (such as a smartphone) (Step 1). The Movement Conformance Engine may direct the user to place the phone in a particular location and confirm that the user's current physical environment provides enough space for the user to perform one or more exercises. In other embodiments, the computing device may be affixed/mounted on the user's body. For example, the user may hold the computing device as the user performs the one or more exercises.

The measurement pipeline prepares a user to perform one or more exercises. (Step 2) In various embodiments, the Movement Conformance Engine may guide the user by providing coaching prompts to instruct the user in how to obtain one or more key postures of an exercise(s). Movement Conformance Engine may further record video data as the user responds to the coaching prompts in order to determine the user's baseline performance and/or ability to conform to the key postures.

The measurement pipeline prompts the user to initiate the one or more exercises and generates data representative of the user's performance of the exercises. (Step 3) according to various embodiments, the Movement Conformance Engine may prompt the user to attempt to perform as many repetitions of the particular exercise within a given amount of time. During the user's performance, the Movement Conformance Engine generates video data in real-time and prompts to be provided to the user based on analysis of the video data in order to assist the user in the performance of the exercise. In addition, the measurement pipeline prompts the user when the exercise is complete. (Step 4)

The measurement pipeline validates whether the user's performance of the exercises is conformant. (Step 5) In various embodiments, the Movement Conformance Engine determines orientation changes that occurred at various parts of the user's body during performance of the exercise. For example, the Movement Conformance Engine determines changes to an armature representation of the user performing the exercise. The Movement Conformance Engine compares the determined armature changes to expected changes associated with an ideal performance of the exercise.

The measurement pipeline stores data associated with the user's performance of the exercises. (Step 6) and executes post-processing of the data. (Step 7) In various embodiments, the Movement Conformance Engine compares the determined changes to the user's baseline performance and/or performance statistics related to one or more segments of types of users to determine whether the user is improving and whether the user's performance of the exercise is conformant.

Various embodiments of the Movement Conformance Engine capture video of a user performing an exercise and inputs video frames in a machine learning model. The Movement Conformance Engine receives from the machine learning model an incoming stream of armatures, whereby each armature corresponds to a particular video frame. For example, given a video frame of a user performing various physical movements, the machine learning model returns a corresponding armature of select points on and within the portrayal of the user's body in the video frame. The select point may be positioned to indicate various types of body joints.

In various embodiments, The machine learning model may be based at least in part on a stock PoseNet network trained on MPII Human Poses dataset using the Caffe framework. The machine learning model may also be based at least in part on a CocoNet version results in a similar though not entirely identical set of keypoints. Any 2D or 3D pose estimation models may also be used similarly.

For various embodiments described herein, the Movement Conformance Engine determines a conformance of a user's particular performance of a given exercise. To determine conformance, the Movement Conformance Engine compares the user's movements captured in video frame against data representative of a standard or idealized movement. Conformance is contingent on or indicative of the patient's medical (musculoskeletal) state. The Movement Conformance Engine further utilizes repetition (“rep”) counts and armature configurations related to a user's particular performance of a given exercise to determine a given band of conformance with respect to a given exercise or sequence of exercises performed by the user.

Rep counts are not only defined by the Movement Conformance Engine as counted completion of an instance of an exercise, but may further be defined as a detected rate at which a user is able to achieve a target posture from some initial position. A band of conformance, or range of conformance, describes an armature and/or rep count that is comparable with a defined population of users. For example, given a user's actual armature and/or rep count, the Movement Conformance Engine classifies the user's performance into a conformance band. For example, an arm placed overhead by the user during performance of a particular exercise that is detected as failing to reach an ideal vertical extension is determined by the Movement Conformance Engine as being in a lower conformance range whereas a more extended reach that is completely vertical is in a higher conformance range. In addition, conformance determined by the Movement Conformance Engine may further contain a description of various armatures that are non-compliant and/or unsafe.

According to various embodiments described herein, an exercise may be a sequence of movements to be performed. The Movement Conformance Engine decomposes video frames of a user performing an exercise on a keyframe basis. A rep-count of an exercise movement is defined by the Movement Conformance Engine as having a starting posture, 0-N intermediate postures and a target posture. Each posture has ranges of conformance, as in a yoga pose in which a beginner will have a lesser reach and also perhaps more time needed to reach, whereas a more proficient practitioner would have a greater reach and speed. In addition, there are conformance rules (or matching rules) applied between each keyframe posture, that describe conformance as between postures in keyframes.

According to various embodiments described herein, the Movement Conformance Engine models an exercise as a sequence of posture keyframes and posture tweens. The Movement Conformance Engine identifies a posture being performed by the user based on a skeleton-like armature with labeled joint indicators and a set of matching rules for matching an input armature (based on video of the user's physical movements) to that armature, and some actions to be taken by the Movement Conformance Engine once the input armature is matched or if the armature is determined to be non-compliant.

As such, various embodiments process an exercise as an ordering of one or more tweens and key postures with a pointer to a current step in a program counter. The Movement Conformance Engine further includes matching rules for determining whether input armatures for a user's physical movements portrayed in video frames match to data that represents a conformant performance of the exercise or a non-compliant performance of the exercise.

According to various embodiments, the Movement Conformance Engine obtains 3D data from combined use of camera, lidar, and machine learning model to obtain a 3D position of a smartphone at a point in time. The Movement Conformance Engine thereby obtain an absolute 3D position of the smartphone in time, from which velocity can be determined and visualized and the average acceleration inferred as well.

Various embodiments include a module(s) and/or one or more functionalities to redact privacy information/data, to encrypt information/data and to anonymize data to ensure the confidentiality and security of user data, user health data and platform information/data as well as compliance with data privacy law(s), health data management laws and regulations in the United States and/or international jurisdictions.

In various embodiments, the Movement Conformance Engine may be implemented via a cloud computing software platform that receives and sends data to one or more software modules executed on a remote computing device, such as a smartphone. In some embodiments, a machine learning network(s) may be accessible at the cloud computing software platform and further may also be executed on the remote computing device according to the one or more software modules. It is understood that any of the operations, steps, methods, processing, data capture, data generation and/or data presentation described herein may occur at the cloud computing software platform, on the remote computing device and/or distributed between the cloud computing software platform and the remote computing device.

In some embodiments, a user may be a medical patient recovering from a surgery and/or an injury. A computing device (or computer device), such as a smart phone computing device, may be situated at the user's body while the user is instructed to perform one or more predefined movements. The computing device tracks and records movement data representative of the user's performance of the predefined movement. During performance of the predefined movement, the Movement Conformance Engine may provide feedback to the user. The Movement Conformance Engine may calculate various statistics and measures in order to determine a quality of the performance by the user and/or a degree of health improvement of the user and/or a comparison of the user's current physical condition with respect to a representative sample of similarly situated medical patients based at least in part on the user's performance(s) of the predefined movement.

According to some embodiments, the Movement Conformance Engine converts movement data into image data, wherein the movement data is associated with a motion sensor (such as an accelerometer) of the computing device situated at the user's body during performance of the predefined movement. The Movement Conformance Engine creates machine learning input based on the image data (i.e. converted movement data) and feeds the input into a machine learning network(s). The Movement Conformance Engine receives output from the machine learning input and determines one or more actions based at least in part on the received machine learning output. In various embodiments, the output from the machine learning network may represent a degree of conformity of the user's performance of the predefined movement with a threshold degree of conformity.

In various embodiments, the user may be holding the computing device while performing a predefined movement(s). The user may be further providing input during performance of the predefined movement and the Movement Conformance Engine captures such input. For example, such input may be audio input and/or input generated by user interface selections (such as finger swipe gestures) applied to a user interface associated with the Movement Conformance Engine.

The Movement Conformance Engine correlates the user-provided input with the movement data. For example, the user may be a medical patient recovering from shoulder surgery who holds a smartphone in her hand and performs a predefined movement(s) with her arm. As she performs the predefined movement, the user may continuously have her finger(s), such as her thumb, in contact with a user input region of the smartphone (such as the smartphone's touchscreen). The user may perform various types of input gestures with her thumb as she progresses through the predefined movement(s). A first type of input gesture may indicate the user experiences a certain degree of pain and/or difficulty and a subsequently received second type of input gesture may indicate the user experiences a different degree of pain and/or difficulty.

In some embodiments, the computing device may be situated at a distance away from the user's body. The computing device may have one or more cameras to capture image and/or video data of the user's body. The Movement Conformance Engine implements one or more computer vision techniques in order to generate a skeletal armature representation of the user's body. The Movement Conformance Engine further captures image and/or video data of the user's performance of the predefined movement(s) and estimates movement of the skeletal armature representation to update a display of the user's skeletal armature representation. The Movement Conformance Engine creates machine learning input based on the estimated movement of the skeletal armature representation and feeds the input into a machine learning network(s). The Movement Conformance Engine receives output from the machine learning network(s) and determines one or more actions based at least in part on the received machine learning output.

Further areas of applicability of the present disclosure will become apparent from the detailed description, the claims and the drawings. The detailed description and specific examples are intended for illustration only and are not intended to limit the scope of the disclosure.

### BRIEF DESCRIPTION OF THE DRAWINGS

The present disclosure will become better understood from the detailed description and the drawings, wherein:

FIG. 1 is a diagram illustrating an exemplary environment in which some embodiments may operate.

FIG. 2 is a diagram illustrating an exemplary environment in which some embodiments may operate.

FIG. 3 is a diagram illustrating an exemplary method that may be performed in some embodiments.

FIG. 4 is a diagram illustrating an exemplary method that may be performed in some embodiments.

FIG. 5 is a diagram illustrating an exemplary method that may be performed in some embodiments.

FIG. 6 is a diagram illustrating an exemplary environment in which some embodiments may operate.

FIG. 7 is a diagram illustrating an exemplary environment in which some embodiments may operate.

FIG. 8 is a diagram illustrating an exemplary environment in which some embodiments may operate.

FIG. 9 is a diagram illustrating an exemplary environment in which some embodiments may operate.

FIGS. 10A and 10B are each a diagram illustrating an exemplary environment in which some embodiments may operate.

FIG. 11 is a diagram illustrating an exemplary environment in which some embodiments may operate.

FIG. 12 is a diagram illustrating an exemplary environment in which some embodiments may operate.

### DETAILED DESCRIPTION

In this specification, reference is made in detail to specific embodiments of the Movement Conformance Engine. Some of the embodiments or their aspects are illustrated in the drawings.

For clarity in explanation, the Movement Conformance Engine has been described with reference to specific embodiments, however it should be understood that the Movement Conformance Engine is not limited to the described embodiments. On the contrary, the Movement Conformance Engine covers alternatives, modifications, and equivalents as may be included within its scope as defined by any patent claims. The following embodiments of the Movement Conformance Engine are set forth without any loss of generality to, and without imposing limitations on, the claimed Movement Conformance Engine. In the following description, specific details are set forth in order to provide a thorough understanding of the present Movement Conformance Engine. The present Movement Conformance Engine may be practiced without some or all of these specific details. In addition, well known features may not have been described in detail to avoid unnecessarily obscuring the Movement Conformance Engine.

In addition, it should be understood that steps of the exemplary methods set forth in this exemplary patent can be performed in different orders than the order presented in this specification. Furthermore, some steps of the exemplary methods may be performed in parallel rather than being performed sequentially. Also, the steps of the exemplary methods may be performed in a network environment in which some steps are performed by different computers in the networked environment.

Some embodiments are implemented by a computer system. A computer system may include a processor, a memory, and a non-transitory computer-readable medium. The memory and non-transitory medium may store instructions for performing methods and steps described herein.

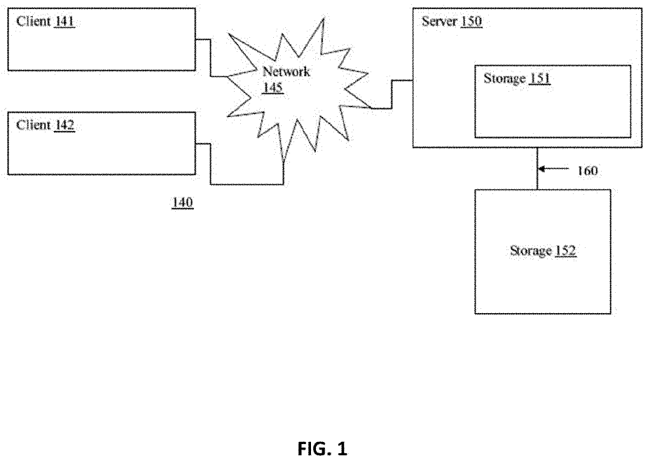

A diagram of exemplary network environment in which embodiments may operate is shown in FIG. 1. In the exemplary environment 140, two clients 141, 142 are connected over a network 145 to a server 150 having local storage 151. Clients and servers in this environment may be computers. Server 150 may be configured to handle requests from clients.

The exemplary environment 140 is illustrated with only two clients and one server for simplicity, though in practice there may be more or fewer clients and servers. The computers have been termed clients and servers, though clients can also play the role of servers and servers can also play the role of clients. In some embodiments, the clients 141, 142 may communicate with each other as well as the servers. Also, the server 150 may communicate with other servers.

The network 145 may be, for example, local area network (LAN), wide area network (WAN), telephone networks, wireless networks, intranets, the Internet, or combinations of networks. The server 150 may be connected to storage 152 over a connection medium 160, which may be a bus, crossbar, network, or other interconnect. Storage 152 may be implemented as a network of multiple storage devices, though it is illustrated as a single entity. Storage 152 may be a file system, disk, database, or other storage.

In an embodiment, the client 141 may perform the method 200 or other method herein and, as a result, store a file in the storage 152. This may be accomplished via communication over the network 145 between the client 141 and server 150. For example, the client may communicate a request to the server 150 to store a file with a specified name in the storage 152. The server 150 may respond to the request and store the file with the specified name in the storage 152. The file to be saved may exist on the client 141 or may already exist in the server's local storage 151. In another embodiment, the server 150 may respond to requests and store the file with a specified name in the storage 151. The file to be saved may exist on the client 141 or may exist in other storage accessible via the network such as storage 152, or even in storage on the client 142 (e.g., in a peer-to-peer system).

In accordance with the above discussion, embodiments can be used to store a file on local storage such as a disk or on a removable medium like a flash drive, CD-R, or DVD-R. Furthermore, embodiments may be used to store a file on an external storage device connected to a computer over a connection medium such as a bus, crossbar, network, or other interconnect. In addition, embodiments can be used to store a file on a remote server or on a storage device accessible to the remote server.

Furthermore, cloud computing is another example where files are often stored on remote servers or remote storage systems. Cloud computing refers to pooled network resources that can be quickly provisioned so as to allow for easy scalability. Cloud computing can be used to provide software-as-a-service, platform-as-a-service, infrastructure-as-a-service, and similar features. In a cloud computing environment, a user may store a file in the “cloud,” which means that the file is stored on a remote network resource though the actual hardware storing the file may be opaque to the user.

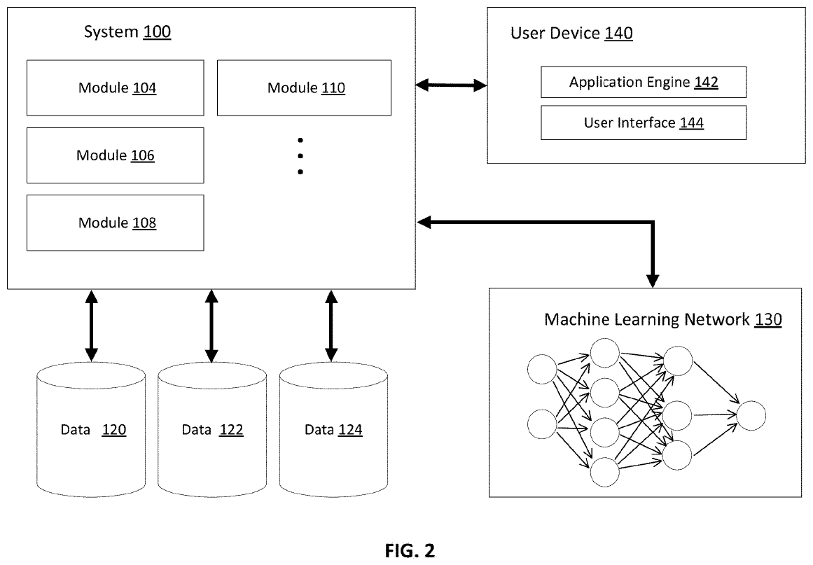

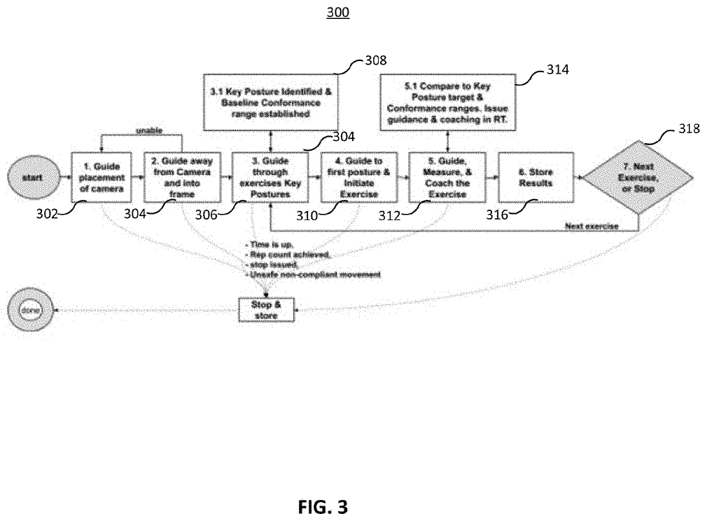

FIG. 2 illustrates a block diagram of an example system 100 for a Movement Conformance Engine that includes respective modules 104, 106, 108, 110 . . . for initiating, implementing and executing any of the operations, steps, methods, processing, data capture, data generation and/or data presentation described herein and illustrated by any of FIGS. 3, 4, 5, 6, 7, 8, 9, 10A, 10B and/or 11. The system 100 may communicate with a user device 140 to display output, via a user interface 144 generated by an application Movement Conformance Engine 142.

While the databases 120, 122 and 124 are displayed separately, the databases and information maintained in a database may be combined together or further separated in a manner the promotes retrieval and storage efficiency and/or data security. It is understood that, in various embodiments, one or more of the modules 104, 106, 108, 110 . . . may reside and be implemented on the user device 140. In addition, respective portions of one or more of the modules 104, 106, 108, 110 . . . may reside and be implemented on the user device 140, while other portions of the same one or more modules 104, 106, 108, 110 . . . may reside and be implemented remotely from the user device 140.

As shown flowchart 300 of FIG. 3, the Movement Conformance Engine instructs the user to place a computing device in a location such that a camera(s) associated with the computing device can capture video/images of the user performing various types of exercises. (Act 302) In various embodiments, the Movement Conformance Engine may include voice and/or user interface prompts providing instructions to the user to place the computing device at a certain location or when to place the device at a certain location. It is understood that the embodiments described herein may allow for the user to hold the computing device during performance of an exercise(s) as opposed to placing it at a particular location.

In some embodiments, the Movement Conformance Engine captures accelerometer and/or gyroscope data to determine whether the computing device is positioned in a proper orientation (e.g. vertical orientation, horizontal orientation). In various embodiments, the Movement Conformance Engine executes one or more machine learning models and/or augmented reality (AR) processing to detect and/or recognize characteristics of the physical environment of the user to determine whether the user has enough open space available to perform one or more exercises.

The Movement Conformance Engine further provides prompts and/or instructions to guide the user away from the placed computing device and to select a placement for themselves in relation to the placed computing device such that the user may be captured by the camera and images of the user displayed on the user interface displayed by computing device is visible to the user. (Act 304)

In various embodiments, if the physical environment surrounding the user is such that the user cannot comply with the Movement Conformance Engines prompts to reach a suitable or comfortable placement at a particular distance away from the computing device, the user may provide input data indicating the user is unable to comply with the Movement Conformance Engines prompts. In such a scenario, according to various embodiments, the Movement Conformance Engine may return to Act 302 in order to assist the user in setting the computing device in an alternate location such that the user may be subsequently able to comply with the prompts in Act 304.

The Movement Conformance Engine may display on the user interface on the computing device a series of key postures that are to be performed as part of an exercise(s) in order to guide the user through a correct and/or accurate performance of the exercise. (Act 306) During such guidance, the Movement Conformance Engine captures image data via the camera of the user performing (or attempting to perform) each key posture. As the user performs the key postures, the Movement Conformance Engine generates data representing various points and/or portions of the user's body with respect to conformant performance of the key postures in order to establish a baseline conformance range for the user. (Act 308)

According to various embodiments, for each key posture, the Movement Conformance Engine may display a guide armature that portrays a conformant performance of a key posture and may provide prompts and/or instructions requesting the user attempt to align a real-time image of their body displayed on the computing device with the displayed armature.

The Movement Conformance Engine sends video frames of the user's physical movements to a machine learning model that returns an incoming stream of armatures that correspond to the user and compares respective positions of the user's body joints and body portions represented in the user's armature with various armatures representing conformant performance of the key posture. In various embodiments, the user may provide voice input indicating a degree of effort and/or pain the user is experiencing at any given moment.

Upon capturing the user's baseline conformance range for the key postures, the Movement Conformance Engine guides the user to a starting position of an initial key posture of a first exercise. (Act 310) For example, the Movement Conformance Engine may provide audio and/or user interface prompts indicating the starting position (or starting key posture) that the user should attempt to perform. In some embodiments, the user may provide an input command, such as voice input data, indicating that the user has begun (or is ready to begin) the exercise(s).

The Movement Conformance Engine triggers initiation of the exercise(s) and displays an armature performing a series of the key postures. (Act 312) In various embodiments, the Movement Conformance Engine captures image data of the user's performance of the key postures and generates an armature of the user based on the user's real-time physical movements and may output various types of audio and/or graphic coaching prompts based on real-time analysis of the joints and connection positions of the user's armature in comparison with a guide armature for a key posture(s). (Act 314) In various embodiments, the Movement Conformance Engine may execute one or more matching rules (further described herein) to generate output from comparison between the user's armature and the one or more guide armature for respective key postures.

Upon completion of a particular exercise, the Movement Conformance Engine may store results from the analysis of the user's armature via the matching rules. (Act 316). In some embodiments, the results (or a portion of the results) may be sent to a cloud computing platform for further analysis, such as, for example, analysis via one or more machine learning models. In some embodiments, a portion of the results may also be analyzed according to one or more pre-defined calculations locally executed on the computing device. The Movement Conformance Engine may further instruct the user to initiate another series of key postures for a subsequent exercise or the Movement Conformance Engine may terminate the exercise session. (Act 318).

In various embodiments, the Movement Conformance Engine captures physical parameters related to the user's armature over time and uploads the captured data to a processing and storage system. Immediate results from various types of analysis are communicated to the user, such as rep count, and other measures. Such immediate results may be determined by analysis and processing that occurs locally on the computing device.

In various embodiments, the video may be uploaded to a cloud computing system for further analysis as well. For example, the Movement Conformance Engine may upload the user's armature(s) and data representing armature changes from video frame-to-video frame. Various modules of the Movement Conformance Engine may be implemented on the cloud computing system to perform calculation with response to the armature changes, including (but not limited to): an amount of movement of each limb, a length of each ‘limb,’ and/or angular changes to each joint. Such angular changes may be, for example, angular velocity. Various types of calculated armature changes may further be represented as vectors and used by the Movement Conformance Engine within further machine learning models.

The Movement Conformance Engine may utilize a pool of data based at least in part on calculated anonymized armature changes from numerous users to determine standards for movement across various population segments. Such standards may be further based at least in part on coincident anonymized user data such as age, comorbidities, and conditions. From machine learning models and analytical methods, the Movement Conformance Engine may derive further changes to an exercise regime for any user as well as adjustments made to conformance range and/or rep count target of subsequent iterations of an exercise(s) and/or other exercises prescribed and other content of the system adjusted for the user.

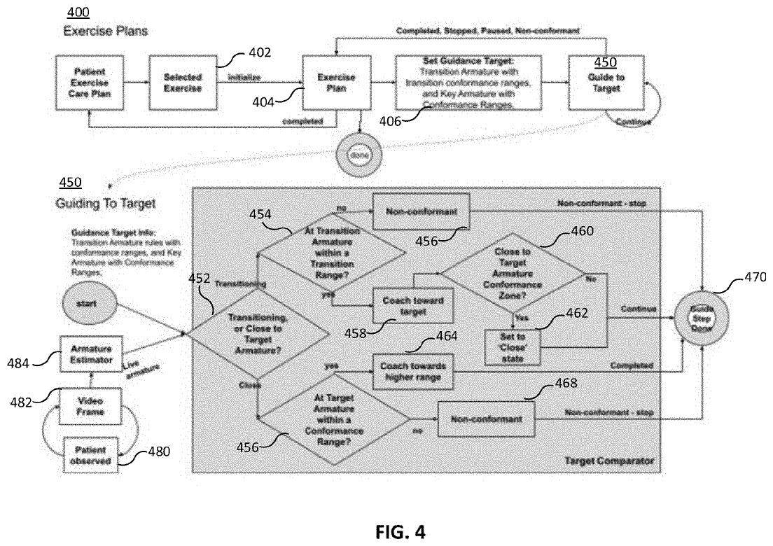

As shown in flowchart 400 of FIG. 4, the Movement Conformance Engine may perform a guided exercise plan(s). The Movement Conformance Engine receives selections of one or more predefined exercises for a patient to perform as part of an exercise care plan. (Act 402). The Movement Conformance Engine initializes an exercise plan based in part on the selected exercise(s). (Act 404) An exercise care plan may describe an exercise(s) the patient should perform according to a certain number of repetitions during a pre-defined amount of time. In some embodiments, the exercise care plan may include multiple sessions of the exercise(s) at different times in order for the Movement Conformance Engine to capture data to determine a rate of health improvement of the patient.

As shown in flowchart 450 of FIG. 4, the Movement Conformance Engine receives an incoming stream of armatures representative of the user's various physical movements portrayed in video frames. The Movement Conformance Engine generates the video frames (Act 482) based on the camera of the computing device recording the user's physical movements. (Act 480). The Movement Conformance Engine feeds the video frames into a pose model which returns an incoming stream of data representative of the user's armatures and/or changes to one or more portions of the user's armatures. The Movement Conformance Engine utilizes the various instances of the user's armatures to determine whether the user's performance of the physical movements are conformant.

The Movement Conformance Engine guides the user in performing one or more physical movements such that the user's armature matches a target armature (or guide armature). The Movement Conformance Engine determines whether the user's armature matches (or is within a range close to matching) the target armature. (Act 452) The Movement Conformance Engine determines whether the user's armature represents that the user's performance is within a conformance range. (Act 456) If not, then the Movement Conformance Engine stops guiding the user towards the target armature. (Acts 468, 470) If within the conformance range, the Movement Conformance Engine provides coaching prompts to the user to motivate the user to improve their current performance in order to increase the measure of the user's conformance. (Act 464) The Movement Conformance Engine determines that the user's armature represents that the user has completed performance of a physical movement(s) and stops further guidance. (Act 470)

In addition, the Movement Conformance Engine confirms whether data related to transitions between instances of the user's armatures matches (or is within a range close to matching) an expected range of armature transition data. (Act 454) If the transition data is not within an expected transition range, the Movement Conformance Engine stops guiding the user towards the target armature. (Acts 456, 470) If the transition data is within the expected transition data range, the Movement Conformance Engine provides coaching prompts to the user to motivate the user to improve their current performance. (Act 458) The Movement Conformance Engine determines whether the user's armature represents a performance of the physical movement that is within a conformance range for the target armature. (Act 460) If so, the Movement Conformance Engine sets a close state (Act 462), sets a transition state of continue, and stops further transition guidance. (Act 470) The guidance step (Act 450) transitions with continue back to itself, whereupon the close state is utilized (Act 452, Paragraph #65 above)

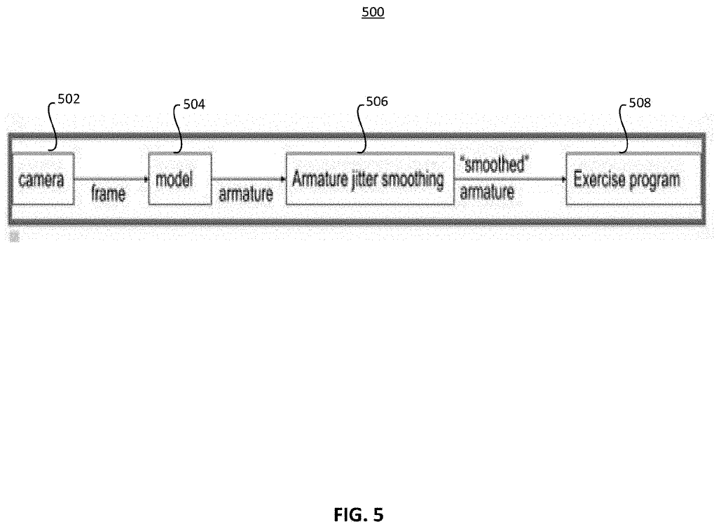

As shown in the flowchart 500 of FIG. 5, a camera(s) associated with the Movement Conformance Engine may continually capture image data, such as one or more image frames, of a user performing a predefined exercise(s). (Act 502) The Movement Conformance Engine may send as input the one or more image frames into a machine learning model in order to continually generate a skeletal armature representation of the user and various updates to the user's skeletal armature representation. (Act 504) As the Movement Conformance Engine generates data for rendering and displaying various instances of the skeletal armature representation of the user in the image data, the Movement Conformance Engine applies one or more averaging smoothing algorithms on one or more of the image frames. (Act 506) As a result of the averaging smoothing algorithms, the Movement Conformance Engine generates image frames with “smoothed” instances of the user's skeletal armature representation for display on a user interface as the user performs one or more movements of a predefined exercise during a set amount of time. (Act 508)

In various embodiments, the Movement Conformance Engine receives video frames generated by a computing device's camera(s) with image data portraying the user performing an exercise(s). The Movement Conformance Engine sends the received frames to an armature model module which returns an armature representing locations of body joints and connections of the user as they are portrayed in the video frames. The armature model may reside in a cloud computing environment, locally on the computing device or be distributed across the cloud computing environment and the computing device. In some embodiments, the armature model may be based on open source software.

In various embodiments, the armature model may return subsequent armatures that correspond to the user from video frame to video frame whereby the received armatures may include recognition jitter. In some embodiments, recognition jitter results from the armature model returning armatures that are inaccurate and/or fail to represent consistent transitions of locations of particular joints and connections between successive armatures generated for a series of video frames. The Movement Conformance Engine implements a smoothing stage to the incoming stream of armatures received from the armature model in order to remove, correct, update and/or alter one or more respective armatures so as to remove recognition jitter.

In order to smooth the incoming stream of armatures, embodiments of the Movement Conformance Engine capture data from multiple armatures from the incoming stream and determines data averages for various body joints and/or connections over time. For example, the Movement Conformance Engine aggregates armatures that correspond to multiple video frames of the user performing an exercise(s) and applies a prioritization algorithm in order to give a different importance weight to various armature joints and/or connections in a set of armatures received during a particular window of time. The Movement Conformance Engine performs various types of averaging functions across the set of armatures to obtain successive armatures that include consistent and “smooth” location changes for joint and connections between successive armatures.

In various embodiments, the smoothing stage implemented by the Movement Conformance Engine includes physiological correction filtering. Some recognition jitter may occur when a PoseNet model swaps certain armature keypoints on the armature, such swapping joint indicators that correspond to the left and right knees. For example, in a first frame, a left hip joint indicator of a first armature connects to a left knee joint indicator which in turn connects to left ankle joint indicator, and similarly for the right joints. However, a second armature, which corresponds to an adjacent successive video frame, identified by the PoseNet model may include the left knee joint indicator where the right knee joint indicator should be—and the right knee joint indicator where the left knee joint indicator should be. Thus, the second armature may include one or more inaccuracies resulting from the swapped joint indicators. For example, a first inaccuracy may be that the left hip joint indicator in the second armature connects to the right knee joint indicator.

For physiological correction filtering, the Movement Conformance Engine defines a filtering window over a number (“N”) of video frames. For respective armature left keypoints (i.e. joint indicators for the left shoulder, left elbow, left wrist, left hip, left knee, left ankle) and respective armature right keypoints (i.e. joint indicators for the right shoulder, right elbow, right wrist, right hip, right knee, right ankle) we track whether their positions along an x-axis are to the left or to the right of the axis of symmetry for the body. In various embodiments, the positions may be based on joint indicator pixel positions given in the armature returned by the PoseNet model. The Movement Conformance Engine defines a dominant orientation to represent a most common orientation with respect to the axis of symmetry over the last N frames (i.e. either left or right).

For a current video frame, the Movement Conformance Engine enforces the orientation given by the dominant position on the left and right keypoints of the corresponding armature returned by the PoseNet model. For example, in some embodiments, the dominant orientation may indicate that left keypoints are to the right of the axis of symmetry and the right keypoints are to the left of the axis of symmetry. For example, the Movement Conformance Engine may detect that an armature corresponding to a current video frame portrays a left knee joint indicator to the left of the axis of symmetry, the Movement Conformance Engine swaps a position of the left knee joint indicator with a position of a right knee joint indicator in order to be in alignment with the dominant orientation.

In various embodiments, the smoothing stage implemented by the Movement Conformance Engine further includes moving average filtering. For any given video frame, the Movement Conformance Engine defines a smoothing window duration of time over N prior video frames. For every joint indicator in a current armature that corresponds with the given video frame, the Movement Conformance Engine adjusts the joint indicator's position based on its average position from the armatures for video frames within the smoothing window. In various embodiments, the Movement Conformance Engine may specifically execute moving average filtering with regards to video frames from the user's performance of a Sit-to-Stand exercise during an exercise time range of 30 seconds.

In various embodiments, the smoothing stage implemented by the Movement Conformance Engine further includes exponentially weighted smoothing. For any given video frame, the Movement Conformance Engine defines a smoothing window duration of time over N prior video frames. The Movement Conformance Engine further defines an exponentially decreasing set of weights during the smoothing window. Exponentially weighted smoothing prioritizes most recently viewed armatures over previously obtained armatures. For example, the Movement Conformance Engine may weight an oldest frame (in time) by e\hat{ }N, a second oldest frame by e\hat{ }(N−1) and so on, until a most recent frame (in time) is weighted by e\hat{ }1.

It is understood that any logarithmic or power relation can be applied and validated empirically. As such, the Movement Conformance Engine normalizes the various frame weights such that a sum of all the frame weights equals 1. To accomplish such normalization, the Movement Conformance Engine divides each weight by e\hat{ }N+e\hat{ }(N−1)+ . . . +e\hat{ }1. For each respective joint indicator in a current armature, the Movement Conformance Engine adjusts the respective joint indicator's position to a position determined according to a weighted average of that respective joint indicator's position in the various armatures within the smoothing window. In various embodiments, the Movement Conformance Engine may implement exponentially weighted smoothing for those types of exercise that generally requires fast movements, such as counting jumping jacks.

In various embodiments, the smoothing stage implemented by the Movement Conformance Engine further includes Savitzky-Golay (“Savgol”) smoothing. The Movement Conformance Engine defines a Savgol filter over the past N frames and applies the Savgol filter to an armature returned by the PoseNet model that corresponds with a current video frame in order to smooth that armature. According to various embodiments, Savgol smoothing emits a frame with a certain amount of time lag (i.e. delay) having processed not only prior samples but subsequent ones.

Note that all smoothing algorithms require N frames to be captured within the smoothing window before smoothing can be used. In cases where frames do not have a fully specified armature due to noise, occlusion of the user, or other factors, the Movement Conformance Engine does not include those frames within the smoothing window, and instead only includes video frames that have corresponding fully specified armatures returned from the PoseNet model.

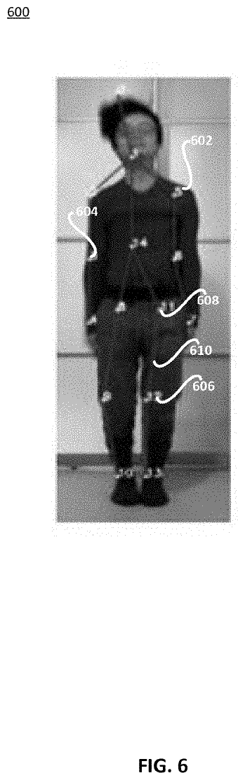

As shown in FIG. 6, the Movement Conformance Engine captures one or more images of a user's current position and stance. For example, a computer device with one or more cameras may be situated remotely from the user in order to capture image data of the user's current position and current stance and subsequent movements. According to various embodiments, the Movement Conformance Engine accesses an armature template that includes one or more joint indicators 602, 604, 606, 608. The Movement Conformance Engine utilizes pixel value data in the images of the user in order to determine where each respective joint indicator 602, 604, 606, 608 is to be displayed with respect to images of the user. In various embodiments, the Movement Conformance Engine may utilize two-dimensional (or three-dimensional) spatial coordinates in place of pixel value data.

The Movement Conformance Engine determines a placement for each respective joint indicator 602, 604, 606, 608 for rendering a skeletal armature representation of the user and subsequent updates to the skeletal armature representation that correspond to the user's subsequent movements. As the user moves and changes a current position and current stance, the Movement Conformance Engine captures additional image data representing the user's movements and tracks changes in the pixel value data that corresponds to each respective joint indicator 602, 604, 606, 608.

According to various embodiments, for example, if the user's left leg moves then the Movement Conformance Engine captures image data representing such movement. The captured image data may include pixel value data, such as a label of a joint indicator located at a particular pixel for the user's left knee as well as other portions of the user's body. For example, the pixel label value data for the user's left knee may be based on the color of the portion of the pants worn by the user that cover the user's left knee. The Movement Conformance Engine may associate the pixel label value and a first pixel region/location where the pixel label value is present in the image data with a left knee joint indicator 606.

According to various embodiments, the captured image data may further include movement of the user's right elbow. The captured image data may represent the pixel label value data for the right elbow as moving from a third pixel region/location to a fourth pixel region/location. As such, the Movement Conformance Engine concurrently updates the skeletal armature representation of the user such that right elbow joint indicator 604 is displayed with respect to the image data at the fourth pixel region/location.

It is understood that as the user's current position and current stance changes due to the user's physical movement, the Movement Conformance Engine continually (or continuously) captures image data and tracks movements of pixel label value data that correspond to the user's joints between various pixel region/locations. The Movement Conformance Engine therefore continually updates a placement and display of the armature joint indicators 602, 604, 606, 608 that correspond with the movement of the pixel label value data. Moreover, a skeletal armature representation of the user may include any number of displayed connections between the respective armature joint indicators 602, 604, 606, 608 and the Movement Conformance Engine may update a length of one or more of the displayed connections based on a changed distance between two joint indicators as measured between updated pixel region/locations. It is further understood that an armature template may have any number of joint indicators and any number of connections between joint indicator pairings. In various embodiments, the Movement Conformance Engine may have multiple different types of armature templates where each armature template is organized with anatomical point that more closely represent a type of movement, a type of exercise, or a stance that occurs during a particular exercise.

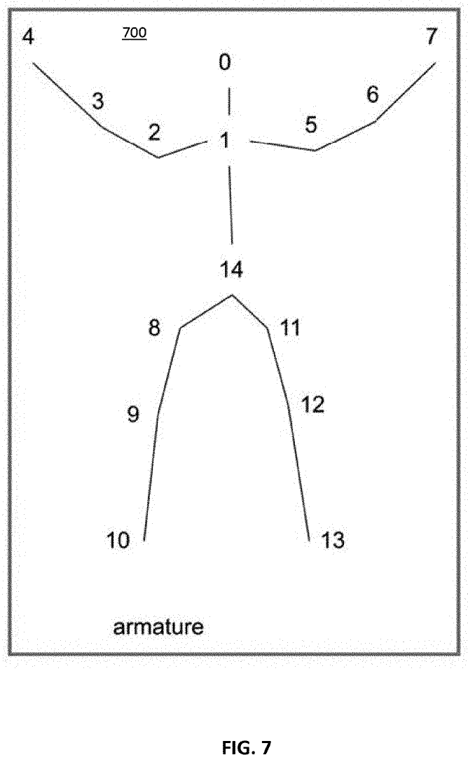

In some embodiments, an armature is a two-dimensional projection of keypoints of the underlying skeleton. For example, an armature may be a MPII-format 15-keypoint armature, where a particular keypoint indicates a position for the user's top of head, neck, left shoulder, left elbow, left wrist, right shoulder, right elbow, right wrist, left hip, left knee, left ankle, right hip, right knee, right ankle and the abdomen. As shown in FIG. 7, an armature 700 of the user may be based on an armature template that includes any number of joint indicators 0, 1, 2, 3, 4, 5, 6, 7, 8, 9, 10, 11, 12, 13, 14. In addition, the armature 700 includes displayed connections between joint indicator pairings. For example, a displayed connection may be a displayed line between two joint indicators 1, 2.

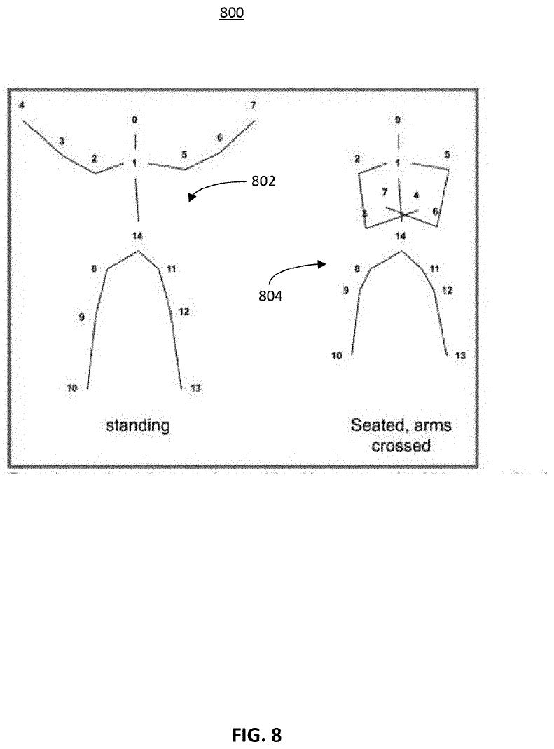

As shown in FIG. 8, the Movement Conformance Engine may have a first armature guide template 802 and a second armature guide template 804. The first armature guide template 802 may represent a beginning position/stance of a predefined exercise and the second armature guide template 804 may represent a terminating position/stance of the predefined exercise or a peak position/stance of the predefined exercise.

The Movement Conformance Engine may instruct a user to perform a predefined exercise where the user is initially positioned according to a position/stance represented by the first armature guide template 802 and attempts to transition to a position/stance represented by the second armature guide template 804 during a predefined time range. For example, the Movement Conformance Engine may concurrently display the user's skeletal armature and the first armature guide template 802 on a user interface. The user may physically move until the user may view joint indicators and displayed connections of the user's skeletal armature as being substantially aligned with the joint indicators and displayed connections of the first armature guide template 802. Once substantially aligned, the user may provide the Movement Conformance Engine with an input command indicating that the user has attempted to achieve the position/stance represented by the first armature guide template 802. For example, when the user can see that the user's skeletal armature is rendered on the user interface in substantial alignment with the first armature guide template 802, the user provides an input command to indicate that the user's current skeletal armature represents the user's performance of the position and stance of the first armature template 802.

In addition, according to various embodiments, the user may also physically move such that the user may view joint indicators and displayed connections of the user's skeletal armature as being substantially aligned with the joint indicators and displayed connections of a display of the second armature guide template 804. Once substantially aligned, the user may provide the Movement Conformance Engine with an input command indicating that the user has attempted to achieve the position/stance represented by the second armature template 804. In addition, the user may provide the Movement Conformance Engine with input indicating a degree of pain and/or difficulty the user is experiencing during a physical movement(s) and the Movement Conformance Engine may include data representative of such user-provided input into any of the determinations and calculations described herein.

Based on receipt of the respective input commands provided by the user, the Movement Conformance Engine captures pixel value data for joint indicators and corresponding lengths of connections of the user skeletal armature. For example, the input commands may be a physical gesture on a touchscreen of a mobile computing device and/or a voice command. Receipt of the input commands from the user indicate to the Movement Conformance Engine that the user has substantially performed a position/stance such that user's skeletal armature is currently displayed in substantial alignment with a displayed armature guide template 802, 804. The Movement Conformance Engine records the placement of the joint indicators, the connections and the connection lengths for the user's skeletal armature as reference points to be used to measure whether the user can perform a predefined exercise in conformance with an expected measure of competency (or conformance).

According to various embodiments, reference points may also be used to estimate how the user's skeletal armature is expected to appear when the user has completed a predefined exercise. For example, if the predefined exercise requires the user to start at the standing position and then end at a seated position, the Movement Conformance Engine may further capture reference points for the user's skeletal armature based on image data portraying the user in a seated position. In various embodiments, the user may cycle through repetitions of a predefined exercise during a set amount of time. Each time the Movement Conformance Engine generates a current version of the user's skeletal armature that aligns with the seated position reference points the Movement Conformance Engine may further count the occurrence of such alignment as an indication that the user has completed a cycle (i.e. rep, count) of the predefined exercise.

According to various embodiments, the Movement Conformance Engine may have access to a set of matching rules that correspond with a predefined exercise(s). For example, the matching rules may represent a plurality of expected armature templates based on positions/stances that occur according to a specific sequence during a correct performance of the predefined exercise(s). The matching rules thereby include data describing armature change relationships between changes in placement of the joint indicators, changes in placements of joint indicator connections and amounts of acceptable changes and angle between connection lengths as well as changes in angles between connections.

According to various embodiments, upon capturing the reference points for the user's skeletal armature, the Movement Conformance Engine may prompt the user to begin performance of a predefined exercise within a set time range. For example, the Movement Conformance Engine may prompt the user to attempt to complete five counts (or reps) of a particular exercise within the set time range. As the user performs the predefined exercise, the Movement Conformance Engine continually analyzes image data of the user's movement to update the joint indicators, connections and connection lengths of the user's skeletal armature. The Movement Conformance Engine continually compares the user's current skeletal armature to the matching rules in order to determine a measure (or degree) of conformity of the user's performance with threshold performance of the predefined exercise as represented by the armature change relationships in the matching rules. In some embodiments, the matching rules may further include rules for transitions between armatures. For example, the matching rule may describe changes that occur during transition from one armature to another armature, where such changes are indicative (or not indicative) of conformance.

For example, the matching rules may indicate that a conforming performance of the predefined exercise may result in a range of acceptable amount of changes of joint indicator locations and acceptable amount of changes to connection lengths between joint indicator pairs as the user's skeletal armature takes on various positions and stances that correspond to the armature change relationships represented by the matching rules. The Movement Conformance Engine compares the user's skeletal armature to the matching rules' armature change relationships in order to determine a degree of conformity between the user's performance.

According to various embodiments, the matching rules may be implemented according to a machine learning network trained on training data representing various segments of individuals performing various predefined exercises. For example, various segments of individuals may include patients within a certain age range, from a particular geographic location, with similar physical limitations, with similar medical problems and/or similar rates of health improvements (i.e. mobility improvements). As such, the Movement Conformance Engine may generate input data based on the user's skeletal armature and feed the input data into a machine learning network. Output from the machine learning network may represent the user's degree of conformity. In various embodiments, the user may select and/or change which segment(s) of individuals the user wishes to be compared against. In addition, output from the machine learning network may include diagnostic output indicating a confirmation of a certain degree of shaking experienced by the user during performance of an exercise whereby such output indicates a likelihood of Parkinson's disease.

According to various embodiments, a user may stand in place while holding a smartphone in the user's right hand. The user extends the right arm while keeping the right arm straight. The user performs a circular motion (or arc motion) with the straight extended right arm while minimizing movement of the user's body and/or torso. The Movement Conformance Engine determines a distance from the right shoulder to the smartphone based on a radius of a theoretical sphere being recorded by the smartphone's motion sensor during the circular motion. As such, the Movement Conformance Engine identifies a position of the right shoulder in three-dimensional space and may utilize the identified position to determine visual placement of a right shoulder joint indicator for the user's skeletal armature.

The Movement Conformance Engine may further instruct the user to hold the smartphone with the right hand bent. Placement of the smartphone as a result of being held by the bent right hand allows for the Movement Conformance Engine to further determine a distance between the right shoulder and the right wrist. Based on the distance between the right shoulder and the right wrist, the Movement Conformance Engine identifies a position of the right wrist in three-dimensional space and may utilize the identified position to determine a visual placement of a right wrist joint indicator for the user's skeletal armature. It is understood the determining a radius with respect to circular motion (or any motion that includes four non-colinear points) of a particular extended body part with respect to the user's still body or torso may be utilized to determine a position in three-dimensional space for a joint indicator for any type of body part on the user's body.

In various embodiments, the Movement Conformance Engine may initiate tracking of circular motion by initially executing a three-dimensional (3D) setup. The Movement Conformance Engine may instruct the user to scan the user's surrounding environment. During the scan, the Movement Conformance Engine captures camera and sensor (i.e. gyroscope, accelerometer) data via the computing device to confirm and/or establish 3D coordinates for a location and/or position of the computing device.

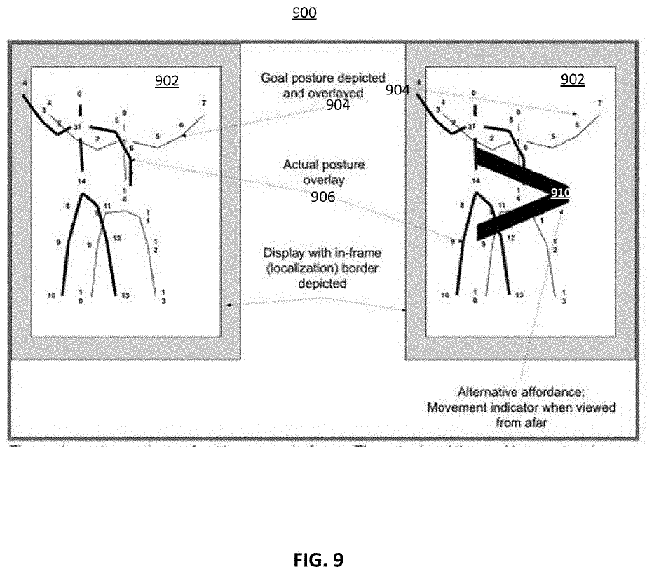

As shown in FIG. 9, the Movement Conformance Engine displays a user interface 902 that includes display of a guide armature 904 and an armature 906 representing a current position and stance of the user. The Movement Conformance Engine displays one or more movement indicators 910 in order to prompt the user to move the user's current position and stance such that the armature 906 rendered by the Movement Conformance Engine and displayed in the user interface 902 may be rendered in alignment with—and overlaid upon—the guide armature 904.

According to various embodiments, aspects illustrated in FIG. 9 may further be utilized for guiding a user into an initial key posture at the outset of performance of an exercise(s). In such a scenario, the Movement Conformance Engine confirms whether an image of the user portrays the user as being completely in frame. In various embodiments, the Movement Conformance Engine may display on the border of the user interface to indicate when the user is in frame in a centered position in the user interface. The Movement Conformance Engine may further modify the color of the border to indicate additional conditions. A certain colored border may be used to indicate, for example, whether the user is position too far away from the camera.

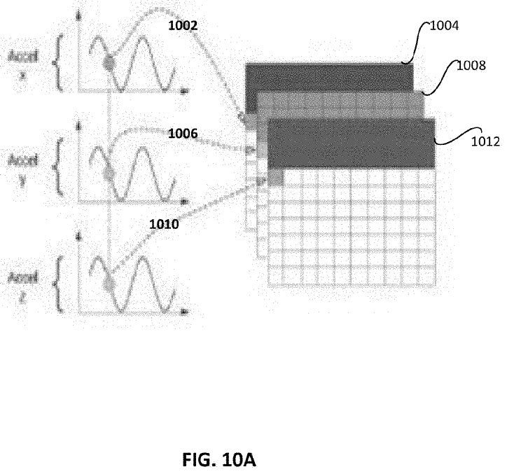

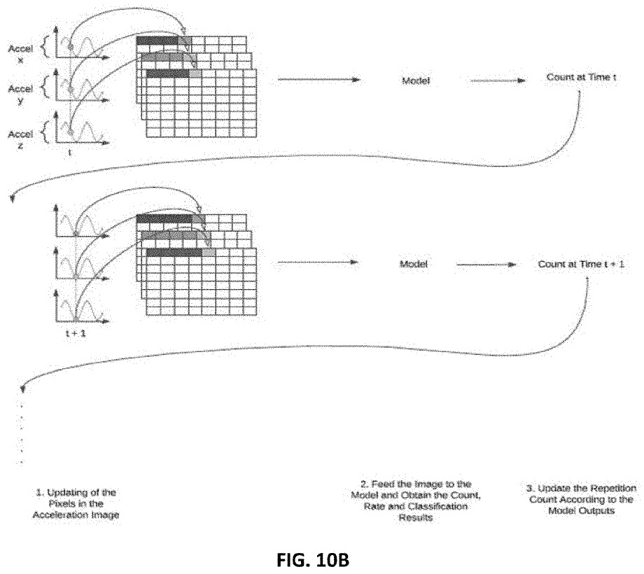

As shown in FIG. 10A, the Movement Conformance Engine converts movement data, such as sensor data, into image data and generates machine learning input data based on the image data. The Movement Conformance Engine feeds the machine learning input data (based on the image date) into one or more machine learning models and machine learning output indicates the user's exercise rep counts and various statistical measures of the user's performance (i.e. conformance, comparisons).

According to various embodiments, a computing device may be held by the user or attached to the user's body while the user performs one or more predefined exercises during a set amount of time. The Movement Conformance Engine captures sensor data from a motion sensor of the computing device. For example, the Movement Conformance Engine captures accelerometer data from one or more accelerometers of the computing device. For example, the sensor data may represent the user's movement according to acceleration values 1002, 1006, 1010 in a three-dimensional space.

The Movement Conformance Engine converts the positional coordinates 1002, 1006, 1010 into image data and further inputs the converted image data into a machine learning network. For example, the various values of the acceleration values 1002, 1006, 1010 may be converted into RGB (red, green, blue) image data. According to various embodiments, each axis (x, y, z) for the positional coordinates 1002, 1006, 1010 may respectively be represented by a specific color. For example, the x axis may be represented by the color red. The y axis may be represented by the color green. The z axis may be represented by the color blue.

In various embodiments, each positional coordinate 1002, 1006, 1010 is a value that reflects a change in velocity (i.e. acceleration). For example, the Movement Conformance Engine captures accelerometer data and the Movement Conformance Engine builds an image based on three color (red, blue, green) that each correspond to a particular x, y, z dimension of the recorded velocity change at a given point in time. Each positional coordinate 1002, 1006, 1010 thereby has a changing value that corresponds to the change in velocity detected by the motion sensor. For example, velocity change coordinate values may fall within the range of 0 to 255. A first value for the z velocity change coordinate 1010 at first moment during the user's movement may be 200 and a second value for the z velocity change coordinate 1010 at a second moment during the user's movement may be 255. Since the color blue corresponds to the z velocity change coordinate 1010, the first value (200) for the z velocity change coordinate 1010 maps to a lighter shade of blue than the second value (255). Values for the x velocity change coordinate 1002 also fall within the range of 0 to 255 but map to varying shades of the color red. Values for they velocity change coordinate 1008 also fall within the range of 0 to 255 but map to varying shades of the color green.

The Movement Conformance Engine builds an image 1004, 1008, 1012 based on pixels that reflect the normalized acceleration coordinates (x, y, z) 1002, 1006, 1010. Each moment during the user's movement that is represented by the sensor data will have corresponding (x, y, z) velocity change coordinate values that map to respective shades of red, green and blue. Each particular moment during the user's movement will correspond to a particular pixel of the image. The Movement Conformance Engine inserts the shades of red, green and blue for the (x, y, z) velocity change coordinate values from a particular moment during the user's movement into the same pixel of the image. For example, if the motion sensor generates motion data during a moment of stillness that occurs during the user's performance of a predefined exercise, then the (x, y, z) velocity change coordinate values for the still moment would be (128, 128, 128). The Movement Conformance Engine determines the shades of red, green and blue that maps to the value of 128. In such a case, all shades of red, green and blue would be grey. As such, the Movement Conformance Engine inserts white into a pixel that corresponds to combined color shades for the (x, y, z) velocity change coordinate values from the still moment during the user's performance of a predefined exercise.

Therefore, an entire image(s) built by the Movement Conformance Engine can be based on shades of red, blue and green to represent an entire performance of a predefined exercise by the user, or an entire movement performed by the user. According to various embodiments, an image built by the Movement Conformance Engine may be rectangular-shaped image with 1800 RGB pixels. The Movement Conformance Engine feeds the converted image data into a machine learning network trained on image data in order to generate output that indicates various attributes and characteristics of the user's movement(s).

According to various embodiments, the user may be holding the computing device in the user's hand during performance of the predefined exercise. The Movement Conformance Engine may receive input data from a touchscreen of the computing device during performance of the predefined exercise and the Movement Conformance Engine may determine a correlation between the received touchscreen input data (or voice input data) with the position and/or position change data. For example, the user may perform various swipe gestures on the touchscreen while performing the predefined exercise. Various types of swipe gestures may be predefined as representing various degrees of difficulty and/or pain currently experienced by the user during performance of the predefined exercise.

The Movement Conformance Engine captures the input data based on the user's swipe gestures and associates the swipe gesture input data with the corresponding (x, y, z) velocity change coordinate values generated by the motion sensor when the swipe gesture input data was received. The Movement Conformance Engine may further generate user interface data to generate a display of a graph presenting changes and relationships between the various degrees of difficulty and/or pain experienced by the user and various periods of conformity or lack of conformity of the user's performance of the predefined exercise. The Movement Conformance Engine may further identify particular time ranges that occurred during the user's performance of the predefined exercise where a threshold degree of conformity regressed to a lack of conformity and may further determine one or more muscle groups being used in the predefined exercise during the identified particular time ranges.

As shown in FIG. 10B, a motion sensor (such as an accelerometer) of a computing device may generate sensor data while a user performs a predefined exercise(s) during a set amount of time. For example, the user may be holding the computing device during performance of the predefined exercise. The motion sensor generates sensor data that the Movement Conformance Engine converts to three-dimensional changes to velocity (x1, y1, z1), (x2, y2, z2), (x3, y3, z3) . . . that represent the computing device's acceleration at a given moment of time during performance of the predefined exercise(s). For example, each respective sensor data may correspond to one second or one millisecond.

The Movement Conformance Engine converts each velocity change coordinate value x1, y1, z1, x2, y2, z2, x3, y3, z3 . . . from a normalizing acceleration RGB value range from 0-255. As illustrated in FIG. 10B, the Movement Conformance Engine builds the image by inserting one pixel at a time, for each respective unit of measurement, according to chronological order. In some embodiments, the image is initialized with (128,128,128) (i.e. velocity change coordinated values that are representative of no movement), and then at time unit 0 the first pixel is added, then at time unit 1, the next pixel is added, and so forth.

In some embodiments, the image space may be large enough that an entire instance of a recorded motion could fill the image space multiple times. In such cases, a pixel for each respective time unit are doubled or tripled. That is, each pixel is inserted into the image by the Movement Conformance Engine twice or three times. For example, video from a 30 second duration that portrays performance of an exercise, sampled at 60 hz, results in an image built with 1800 pixels. In some embodiments, in order to train a machine learning network, such an image provides 1800 different pixel examples to be used as training data.

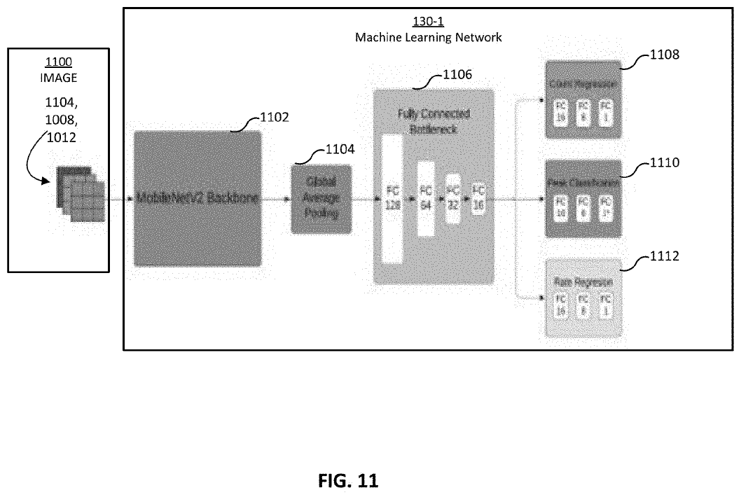

As shown in FIG. 11, the Movement Conformance Engine inserts input into a machine learning network 130-1 trained on various types of image training data. The image training data may be representative of various performances of various types of predefined exercises from one or more individuals that belong to one or more individual segments. The input may be based on image data 1100 built by the Movement Conformance Engine as a result of converting motion sensor data into pixel values representing various shades of red green and blue. According to various embodiments, the machine learning network 130-1 may include a MobileNetV2 Backbone module 1102, a Global Average Pooling module 1104, a Fully Connected Bottleneck module 1106, a Count Progression module 1108, a Peak Classification module 1110 and a Rate Progression module 1112. According to various embodiments, the Count Progression module 1108 may be directed to detecting how many instances or repetitions of a particular predefined exercise performed by the user are represented in the image 1100. The Peak Classification module 1108 may be directed to detecting image data that represents a peak of each instance a particular predefined exercise performed by the user. The Rate Progression module 1112 may be directed to detecting image data that represents a rate at which the user is able to repeat respective cycles or counts of the predefined exercise.

For example, if the predefined exercise requires the user to begin at a seated position and perform movements to arrive at a standing position and then ultimately return to the seated position, the Count Progression module 1108 detects respective instances (i.e. reps, counts) in the image data 1100 that represent the user moving from a seated position through the standing position and back to the seated position. The Peak Classification module 1108 detects image data representing a peak within each count. For example, a peak may be represented by the user reaching the standing position and beginning a descent back to the seated position.

Various embodiments of the Movement Conformance Engine may use any suitable machine learning training techniques to train the machine learning network 130 for each sensor, including, but not limited to a neural net based algorithm, such as Artificial Neural Network, Deep Learning; a robust linear regression algorithm, such as Random Sample Consensus, Huber Regression, or Theil-Sen Estimator; a kernel based approach like a Support Vector Machine and Kernel Ridge Regression; a tree-based algorithm, such as Classification and Regression Tree, Random Forest, Extra Tree, Gradient Boost Machine, or Alternating Model Tree; Naïve Bayes Classifier; and other suitable machine learning algorithms. In some embodiments, multiple types of machine learning models may be used for a particular time range of an exercise(s).

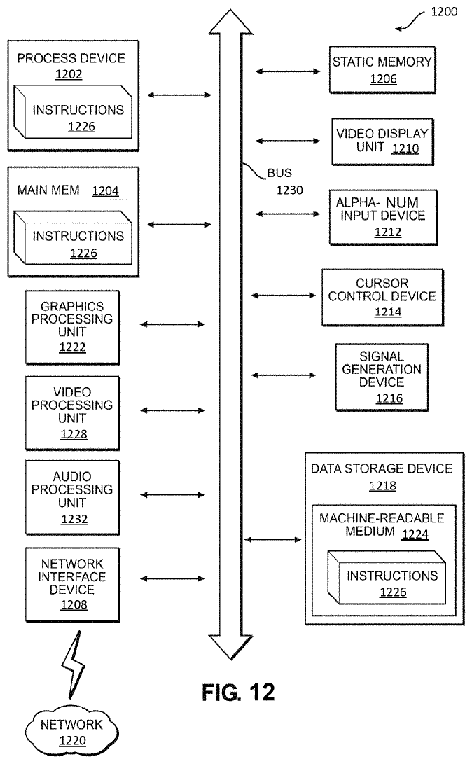

FIG. 12 illustrates an example machine of a computer system within which a set of instructions, for causing the machine to perform any one or more of the methodologies discussed herein, may be executed. In alternative implementations, the machine may be connected (e.g., networked) to other machines in a LAN, an intranet, an extranet, and/or the Internet. The machine may operate in the capacity of a server or a client machine in client-server network environment, as a peer machine in a peer-to-peer (or distributed) network environment, or as a server or a client machine in a cloud computing infrastructure or environment.

The machine may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The example computer system 1200 includes a processing device 1202, a main memory 1204 (e.g., read-only memory (ROM), flash memory, dynamic random access memory (DRAM) such as synchronous DRAM (SDRAM) or Rambus DRAM (RDRAM), etc.), a static memory 1206 (e.g., flash memory, static random access memory (SRAM), etc.), and a data storage device 1218, which communicate with each other via a bus 1230.

Processing device 1202 represents one or more general-purpose processing devices such as a microprocessor, a central processing unit, or the like. More particularly, the processing device may be complex instruction set computing (CISC) microprocessor, reduced instruction set computing (RISC) microprocessor, very long instruction word (VLIW) microprocessor, or processor implementing other instruction sets, or processors implementing a combination of instruction sets. Processing device 1202 may also be one or more special-purpose processing devices such as an application specific integrated circuit (ASIC), a field programmable gate array (FPGA), a digital signal processor (DSP), network processor, or the like. The processing device 1202 is configured to execute instructions 1226 for performing the operations and steps discussed herein.

The computer system 1200 may further include a network interface device 1208 to communicate over the network 1220. The computer system 1200 also may include a video display unit 1210 (e.g., a liquid crystal display (LCD) or a cathode ray tube (CRT)), an alphanumeric input device 1212 (e.g., a keyboard), a cursor control device 1214 (e.g., a mouse), a graphics processing unit 1222, a signal generation device 1216 (e.g., a speaker), graphics processing unit 1222, video processing unit 1228, and audio processing unit 1232.

The data storage device 1218 may include a machine-readable storage medium 1224 (also known as a computer-readable medium) on which is stored one or more sets of instructions or software 1226 embodying any one or more of the methodologies or functions described herein. The instructions 1226 may also reside, completely or at least partially, within the main memory 1204 and/or within the processing device 1202 during execution thereof by the computer system 1200, the main memory 1204 and the processing device 1202 also constituting machine-readable storage media.

In one implementation, the instructions 1226 include instructions to implement functionality corresponding to the components of a device to perform the disclosure herein. While the machine-readable storage medium 1224 is shown in an example implementation to be a single medium, the term “machine-readable storage medium” should be taken to include a single medium or multiple media (e.g., a centralized or distributed database, and/or associated caches and servers) that store the one or more sets of instructions. The term “machine-readable storage medium” shall also be taken to include any medium that is capable of storing or encoding a set of instructions for execution by the machine and that cause the machine to perform any one or more of the methodologies of the present disclosure. The term “machine-readable storage medium” shall accordingly be taken to include, but not be limited to, solid-state memories, optical media and magnetic media.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “identifying” or “determining” or “executing” or “performing” or “collecting” or “creating” or “sending” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

The present disclosure also relates to an apparatus for performing the operations herein. This apparatus may be specially constructed for the intended purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

Various general purpose systems may be used with programs in accordance with the teachings herein, or it may prove convenient to construct a more specialized apparatus to perform the method. The structure for a variety of these systems will appear as set forth in the description above. In addition, the present disclosure is not described with reference to any particular programming language. It will be appreciated that a variety of programming languages may be used to implement the teachings of the disclosure as described herein.

The present disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form readable by a machine (e.g., a computer). For example, a machine-readable (e.g., computer-readable) medium includes a machine (e.g., a computer) readable storage medium such as a read only memory (“ROM”), random access memory (“RAM”), magnetic disk storage media, optical storage media, flash memory devices, etc.

In the foregoing disclosure, implementations of the disclosure have been described with reference to specific example implementations thereof. It will be evident that various modifications may be made thereto without departing from the broader spirit and scope of implementations of the disclosure as set forth in the following claims. The disclosure and drawings are, accordingly, to be regarded in an illustrative sense rather than a restrictive sense.

What is claimed is:

1. A computer-implemented method comprising:
registering a physical location of a computing device with respect to a user in a three-dimensional (3D) space;
collecting movement data of the computing device from the registered location during performance of a predefined movement by the user; and
identifying an action based on at least an attribute associated with the movement data, wherein the movement data can also be used compute velocity at any point and color that point accordingly, AND it could also be fed to a machine learning model for conformance matching, AND/OR rules for matching and determining conformance and performance levels.

2. The computer-implemented method of claim 1, wherein the computing device is further situated at one of:
(i) at a portion of the user's body; and
(ii) at a distance away from the user's body.

3. The computer-implemented method of claim 1, wherein collecting movement data of the computing device from the registered location during performance of a predefined movement by the user comprises:
capturing movement data associated with computing device, the movement data representing one or more changes of a physical orientation of the computing device during performance of the predefined movement by the user resulting in position data and/or change of position as velocity or acceleration;
converting one or more portions of the captured movement data into image data; and
utilizing the converted image data as input to one or more machine learning networks trained at least in part on training image data that corresponds to portrayal of one or more performances of respective predefined physical movements.

4. The computer-implemented method of claim 3, wherein identifying an action based on at least an attribute associated with the movement data comprises:
identifying the action based at least in part on output of the machine learning network.

5. The computer-implemented method of claim 3, wherein converting one or more portions of the captured movement data to image data comprises:
converting accelerometer data into RGB image data, the accelerometer data associated with an accelerometer of the computing device; and
wherein the registered physical location of the computer device corresponds to a location situated at the user's body.

6. The computer-implemented method of claim 1, wherein registering a physical location of a computing device with respect to a user in a three-dimensional (3D) space comprises:
generating a 2D skeletal armature projection of the user's body based on a view of the user's body from a perspective of the computer device; and
wherein the registered physical location of the computer device comprises a location situated at a distance away from the user's body.

7. The computer-implemented method of claim 1, wherein collecting movement data of the computing device from the registered location during performance of a predefined movement by the user comprises:
detecting a change of a position of at least a portion of the skeletal armature representation of the user's body; and
wherein identifying the action based on at least an attribute associated with the movement data comprises:
identifying the action based on at least an attribute associated with the detected change of the position of portion of the skeletal armature representation of the user's body.

8. A system comprising one or more processors, and a non-transitory computer-readable medium including one or more sequences of instructions that, when executed by the one or more processors, cause the system to perform operations comprising:
registering a physical location of a computing device with respect to a user in a three-dimensional (3D) space;
collecting movement data of the computing device from the registered location during performance of a predefined movement by the user; and
identifying an action based on at least an attribute associated with the movement data, wherein the movement data can also be used compute velocity at any point and color that point accordingly, AND it could also be fed to a machine learning model for conformance matching, AND/OR rules for matching and determining conformance and performance levels.

9. The system of claim 8, wherein the computing device is further situated at one of:
(iii) at a portion of the user's body; and
(iv) at a distance away from the user's body.

10. The system of claim 8, wherein collecting movement data of the computing device from the registered location during performance of a predefined movement by the user comprises:
capturing movement data associated with computing device, the movement data representing one or more changes of a physical orientation of the computing device during performance of the predefined movement by the user resulting in position data and/or change of position as velocity or acceleration;
converting one or more portions of the captured movement data into image data; and
utilizing the converted image data as input to one or more machine learning networks trained at least in part on training image data that corresponds to portrayal of one or more performances of respective predefined physical movements.

11. The system of claim 10, wherein identifying an action based on at least an attribute associated with the movement data comprises:
identifying the action based at least in part on output of the machine learning network.

12. The system of claim 10, wherein converting one or more portions of the captured movement data to image data comprises:
converting accelerometer data into RGB image data, the accelerometer data associated with an accelerometer of the computing device; and
wherein the registered physical location of the computer device corresponds to a location situated at the user's body.

13. The system of claim 8, wherein registering a physical location of a computing device with respect to a user in a three-dimensional (3D) space comprises:
generating a 2D skeletal armature projection of the user's body based on a view of the user's body from a perspective of the computer device; and
wherein the registered physical location of the computer device comprises a location situated at a distance away from the user's body.

14. The system of claim 8, wherein collecting movement data of the computing device from the registered location during performance of a predefined movement by the user comprises:
detecting a change of a position of at least a portion of the skeletal armature representation of the user's body; and
wherein identifying the action based on at least an attribute associated with the movement data comprises:
identifying the action based on at least an attribute associated with the detected change of the position of portion of the skeletal armature representation of the user's body.

15. A computer program product comprising a non-transitory computer-readable medium having a computer-readable program code embodied therein to be executed by one or more processors, the program code including instructions to:
registering a physical location of a computing device with respect to a user in a three-dimensional (3D) space;
collecting movement data of the computing device from the registered location during performance of a predefined movement by the user; and
identifying an action based on at least an attribute associated with the movement data, wherein the movement data can also be used compute velocity at any point and color that point accordingly, AND it could also be fed to a machine learning model for conformance matching, AND/OR rules for matching and determining conformance and performance levels.

16. The computer program product of claim 15, wherein the computing device is further situated at one of:
(v) at a portion of the user's body; and
(vi) at a distance away from the user's body.

17. The computer program product of claim 16, wherein collecting movement data of the computing device from the registered location during performance of a predefined movement by the user comprises:
capturing movement data associated with computing device, the movement data representing one or more changes of a physical orientation of the computing device during performance of the predefined movement by the user resulting in position data and/or change of position as velocity or acceleration;
converting one or more portions of the captured movement data into image data; and
utilizing the converted image data as input to one or more machine learning networks trained at least in part on training image data that corresponds to portrayal of one or more performances of respective predefined physical movements.

18. The computer program product of claim 16, wherein identifying an action based on at least an attribute associated with the movement data comprises:
identifying the action based at least in part on output of the machine learning network.

19. The computer program product of claim 15, wherein converting one or more portions of the captured movement data to image data comprises:
converting accelerometer data into RGB image data, the accelerometer data associated with an accelerometer of the computing device; and
wherein the registered physical location of the computer device corresponds to a location situated at the user's body.

20. The computer program product of claim 15, wherein registering a physical location of a computing device with respect to a user in a three-dimensional (3D) space comprises:
generating a 2D skeletal armature projection of the user's body based on a view of the user's body from a perspective of the computer device; and
wherein the registered physical location of the computer device comprises a location situated at a distance away from the user's body.

21. The computer program product of claim 15, wherein collecting movement data of the computing device from the registered location during performance of a predefined movement by the user comprises:
detecting a change of a position of at least a portion of the skeletal armature representation of the user's body; and
wherein identifying the action based on at least an attribute associated with the movement data comprises:
identifying the action based on at least an attribute associated with the detected change of the position of portion of the skeletal armature representation of the user's body.
